(19) United States
(12) Patent Application Publication
(10) Pub. No.: US 2016/0166455 A1
(43) Pub. Date: Jun. 16, 2016

(54) SELF-CONTAINED STERILE SURGICAL ENVIRONMENT

(71) Applicant: Roger F. Steinert, Laguna Beach, CA (US)

(72) Inventor: Roger F. Steinert, Laguna Beach, CA (US)

(21) Appl. No.: 14/965,136

(22) Filed: Dec. 10, 2015

Related U.S. Application Data
(60) Provisional application No. 62/090,923, filed on Dec. 12, 2014.

(51) Int. Cl. A61G 10/00; A61B 42/10; A61F 9/007; A61M 16/10; A61M 16/12; A61G 10/02; A61B 46/23

(52) U.S. Cl. CPC A61G 10/005; A61G 10/023; A61B 42/10; A61B 46/23; A61M 16/105; A61M 16/12; A61F 9/007; A61B 2046/205

(57) ABSTRACT

The present invention relates to a sterile enclosure for performing medical procedures, such as an ophthalmologic surgery. The enclosure includes a lightweight, flexible housing material forming an enclosure, at least one equipment attachment region within the housing material, at least one subject attachment region within the housing material, at least one air intake port, at least one air exhaust port, and at least one user access port within the housing material.

### BACKGROUND OF THE INVENTION

Most current ophthalmologic surgeries occur in open operating theater environments in dedicated surgical suites. These surgical suites are used as a sterile environment to reduce the risk of infection during the procedure. However, the use of such surgical environments to conduct these surgeries can be very inefficient, which may greatly increase the cost and duration of the procedures.

For example, for laser-assisted cataract surgeries, a laser may be used in a traditional non-sterile exam room to make one or more initial incisions and other treatments inside the eye, and then the patient is transported to a dedicated surgical suite for the rest of the procedure. This may increase the risk of infection as well as the duration of the entire procedure, both of which create an increase in direct and indirect costs. Further, using the laser in the surgical suite can tie up an otherwise busy operating room by using time that could be used by other conventional surgeries. Therefore, many short duration ophthalmologic surgeries are inefficient and costly by virtue of their performance within open operating theater environments.

The field of ophthalmologic surgery can therefore be greatly aided with the ability to perform entire surgeries directly in a clean but not sterile room setting. Thus, there is a need in the art for devices, systems and methods for creating a self-contained sterile surgical environment which may be employed for surgeries outside of an open operating surgical theater. The present invention satisfies this unmet need.

### SUMMARY OF THE INVENTION

A sterile enclosure for performing an ophthalmologic procedure is described. The enclosure includes a lightweight, flexible housing material forming an enclosure, at least one equipment attachment region within the housing material, at least one subject attachment region within the housing material, at least one air intake port, at least one air exhaust port, and at least one user access port within the housing material. In one embodiment, the at least one air intake port further comprises a filter. In another embodiment, the at least one access port is a glove integrated with the housing material. In another embodiment, the at least one access port is a flexible septum having separable leaflets. In another embodiment, the flexible housing material includes a frame. In another embodiment, the equipment attachment region includes an adhesive perimeter for at least temporary attachment to the desired equipment component. In another embodiment, the subject attachment region includes an adhesive perimeter for at least temporary attachment around the treatment site of the subject. In another embodiment, the housing material enclosure is inflated with air. In another embodiment, a bactericidal gas is pumped into the housing material enclosure.

Also described is a sterile enclosure for performing a medical procedure. The enclosure includes a lightweight, flexible housing material forming an enclosure, a suspension mechanism for suspending the enclosure above a subject, at least one subject attachment region within the housing material, at least one air intake port, at least one air exhaust port, and at least one user access port within the housing material.

Further, a system for creating a temporary sterile environment to perform an ophthalmologic procedure is described. The system includes a lightweight, flexible housing material forming an enclosure, at least one equipment attachment region within the housing material, at least one subject attachment region within the housing material, at least one user access port within the housing material, and an air flow circuit for controlling the flow of air within the housing material enclosure.

### BRIEF DESCRIPTION OF THE DRAWINGS

The following detailed description of preferred embodiments of the invention will be better understood when read in conjunction with the appended drawings. For the purpose of illustrating the invention, there are shown in the drawings embodiments which are presently preferred. It should be understood, however, that the invention is not limited to the precise arrangements and instrumentalities of the embodiments shown in the drawings.

FIG. 1 is a schematic of an exemplary sterile environment device positioned next to a laser/microscope assembly.

FIG. 2A is a schematic of an exemplary airflow pattern within the sterile environment device of FIG. 1.

FIG. 2B is a front view schematic of the exemplary sterile environment device of FIG. 1.

FIG. 2C is a back view schematic of the exemplary sterile environment device of FIG. 1.

FIG. 3 is a schematic of a surgeon performing an exemplary procedure on a subject's eyes within the sterile environment device of FIG. 1.

FIG. 4 is a schematic of another exemplary sterile environment device positioned over the mid-section of a subject.

### DETAILED DESCRIPTION

It is to be understood that the figures and descriptions of the present invention have been simplified to illustrate elements that are relevant for a clear understanding of the present invention, while eliminating, for the purpose of clarity, many other elements found in typical sterile environment systems. Those of ordinary skill in the art may recognize that other elements and/or steps are desirable and/or required in implementing the present invention. However, because such elements and steps are well known in the art, and because they do not facilitate a better understanding of the present invention, a discussion of such elements and steps is not provided herein. The disclosure herein is directed to all such variations and modifications to such elements and methods known to those skilled in the art.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20%, ±10%, ±5%, ±1%, and ±0.1% from the specified value, as such variations are appropriate.

Throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, 6 and any whole and partial increments therebetween. This applies regardless of the breadth of the range.

As contemplated herein, the present invention includes a single or multi-use device for creating a sterile surgical environment in which to perform surgeries, such as ophthalmologic surgeries, in lieu of an expensive and elaborate operating room. Accordingly, the device not only allows a surgery to be performed at a lower cost basis, but also increases access to patient care without unnecessarily exposing patients to infection and contamination from external environments.

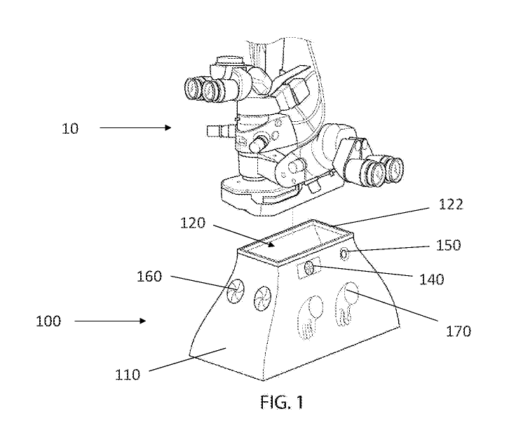

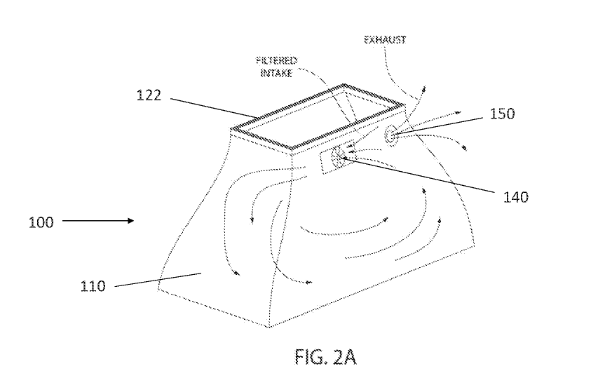

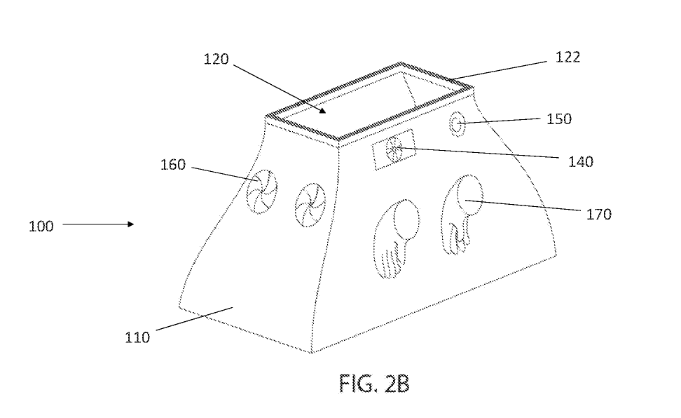

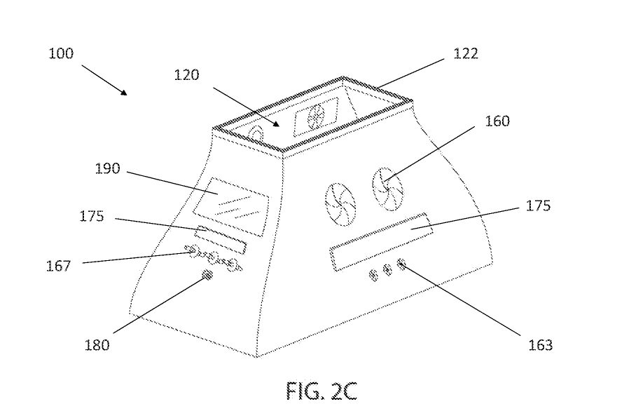

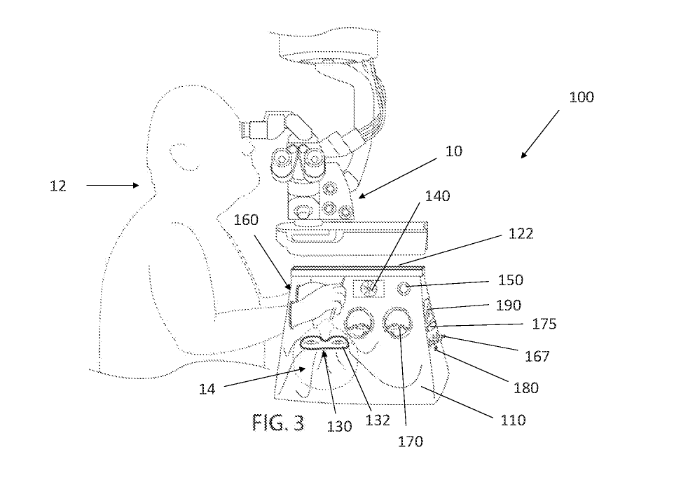

For example, as shown in FIGS. 1 and 3, a first embodiment of the sterile environment device 100 may include an enclosure material 110, at least one surgical tool attachment region 120, at least one subject attachment region 130, at least one gas or air intake port 140, at least one gas or air exhaust port 150, and at least one user access port, such as ports 160 and/or integrated gloves 170. Without limitation, it should be appreciated that device 100 may further include other structural features or openings for suitably introducing additional equipment, gases and the like into the sterile environment, as would be desired. For example, as shown in FIG. 2C, device 100 may include one or more line ports 163 sized to pass standard fluid or gas tubing therethrough, electrical line or other conduit associated with an instrument within the enclosure. In another example, device 100 may include conduit or line engagement ports 167, such as male end fittings to engage fluid or air line tubing inside and/or outside of device 100. Such engagement ports may include any standard interlocking or temporary locking mechanism, and may further include a valve for adjusting flow of a fluid or gas through the engagement port 167. As contemplated herein, line ports 163 and engagement ports 167 may be used for running standard fluid infusion/aspiration handpieces, along with other instrumentation commonly used during cataract surgery. In another example, device 100 may include one or more portals 175 for passing tools, instruments or other device into and out of device 100. Such portals may be of any desired size and shape, and my open and close via a sealable flap, a tilting drawer mechanism, or the like. In another example, device 100 may include one or more electrical outlets or sockets 180 for electrically connecting an instrument or other device within device 100. In another example, when material 110 is not transparent or translucent, device 100 may include one or more viewing regions 190 to promote better viewing of activity within device 100.

Enclosure material 110 may be composed of a flexible and air-impermeable material which may form the walls and generally define the space within the sterile environment device 100. In preferred embodiments, material 110 may be any air-impermeable material that is light and flexible, and more preferably hypoallergenic. In certain embodiments, material 110 may be translucent or transparent to provide at least some visibility through the material and into the sterile environment. As mentioned previously, material 110 may include one or more window regions 190 that are transparent, such that the surgeon may see into the sterile environment through the window. Suitable materials for the enclosure may include any polymeric sheet, woven plastic or other material suitable for providing the desired and described characteristics, as would be understood by those skilled in the art. Preferred materials include but are not limited to polymethymethacralate where rigidity is desired and flexible polymer sheet material, similar to current translucent and transparent surgical drape material, where flexibility is desired. Preferred adhesive materials and adhesive backings include those used with surgical drape materials. Further, enclosure material 110 may be of any desired size and geometry to provide suitable working space within the enclosure of device 100 and to provide adequate ventilation and airflow throughout. For example, the enclosure formed by material 110 may be generally cylindrical, rectangular, spherical or any other shape desired. Preferably, the enclosure may have a generally wider area near the surgical site on the subject.

The enclosure formed from enclosure material 110 may optionally include a frame, having one or more rods, cords, panels or the like, to provide and define the desired shape of the enclosure. Such framing may be integrated within designated folds or looped enclosure material 110, or it may be separable from enclosure material 110 and positioned internally and/or externally to the created sterile enclosure. Such framing may be rigid and/or flexible as desired. Alternatively or additionally, enclosure material 110 may include one or more areas of variable thickness, such that thicker regions may provide additional support or sturdiness to the formed enclosure. Further, enclosure material 110 may include isolated air channels to form inflatable supports for the enclosure when an increased airflow or pressure is introduced into the isolated channels. Further still, enclosure material 110 may include any number of exterior and/or interior pockets for use as either storage compartments, or for insertion of rigid or semi-rigid support panels, as desired.

Surgical tool attachment region 120 may include at least one opening with a perimeter 122 for at least temporary attachment to a surgical tool 10, such as a microscope, surgical laser or any other piece of equipment that must have at least a portion of it exposed to the interior of the sterile enclosure. Multiple attachment regions 120 may be included, if desired. Attachment region 120 may be designed for attachment to any type of surgical tool desired, without limitation. Attachment region perimeter 122 may include an adhesive to permit direct attachment of the enclosure to surgical tool 10. Optionally, a separate component may first be attached to surgical tool 10, and attachment region perimeter 122 then engages the separate component to indirectly and temporarily secure the enclosure to surgical tool 10. As contemplated herein, attachment region 120 may use any attachment mechanism about perimeter 122 as would be understood by those skilled in the art. Preferred attachment systems include adhesive backings, magnets for metallic elements, and slots and grooves for direct connections to fixed elements such as the operating microscope.

Similarly, subject attachment region 130 may include an opening with a perimeter 132 for at least temporary attachment to a subject 14, such that the treatment site of the subject is positioned within perimeter 132 and accessible within the sterile enclosure. Attachment region perimeter 132 may also include an adhesive to permit attachment of the enclosure to subject 14. Optionally, a separate component may first be attached or otherwise positioned about the treatment site of subject 14, such that attachment region perimeter 132 may then engage the separate component to indirectly secure the enclosure to subject 14. As contemplated herein, attachment region 130 may use any attachment mechanism as would be understood by those skilled in the art, such as adhesive backings, magnets for metallic elements, and slots and grooves for direct connections to the subject.

Integrated within enclosure material 110 is at least one air or gas intake port 140 and at least one gas or air exhaust port 150. As shown in FIG. 2B, the intake and exhaust ports 140 and 150 should be positioned within enclosure material 110 such that the air or gases can suitably circulate within the enclosure to provide any necessary wall support of material 110 via inflation by positive air pressures, and/or to maintain sterility via sterilized gasses or bactericidal gases. Intake port 140 may include any desired filter or filtering mechanism to purify incoming air or gas as would be understood by those skilled in the art. Optionally, intake port 140 may include a detachable or integrated rigid component to improve stable positioning of intake port 140 within enclosure material 110, and/or to permit secure engagement of any secondary conduit or the like. Further, intake port 140 may also include any sort of valve or other flow restriction mechanism for adjusting the amount of air entering the interior of the enclosure. Likewise, air exhaust port 150 may include a detachable or integrated rigid component to improve stable positioning of exhaust port 150 within enclosure material 110. Further, exhaust port 150 may also include any sort of valve or other flow restriction mechanism for adjusting the amount of air leaving the interior of the enclosure. As contemplated herein, intake and exhaust ports 140 and 150 may be electrically connected and controlled, such that the regulation of air and other gases within the sterile environment is automated. Further, it should be appreciated that one or more external fans, air pumps, power supplies, processors and any required circuitry may form part of device 100 to reliably supply and control the flow of air and other gases to and from device 100.

Also integrated within enclosure material 110 is at least one access port, such as ports 160 and/or integrated gloves 170. For example, as shown in FIG. 3, a surgeon 12 may insert their hands through access ports 160, such that their hands are available to work within the sterile environment. There is no limitation to the number, size, shape or positioning of ports 160 of device 100. In one embodiment, access ports 160 may be constructed similarly to a flexible septum cut to form leaflets that separate when a user pushes against its surface with suitable force. Optionally, access ports 160 may include a detachable or integrated rigid component to improve stable positioning of access port 160 within enclosure material 110. Use of access ports 160 is preferred when surgeon 12 is wearing sterilized gloves or other suitably sterile hand covering. Alternatively, integrated gloves 170 permit insertion of the surgeon's hands directly into the integrated gloves to introduce them into the sterile environment. Device 100 may include any number of integrated gloves or glove pairs. Integrated gloves 170 may also include a detachable or integrated rigid component to improve stable positioning of integrated gloves 170 within enclosure material 110.

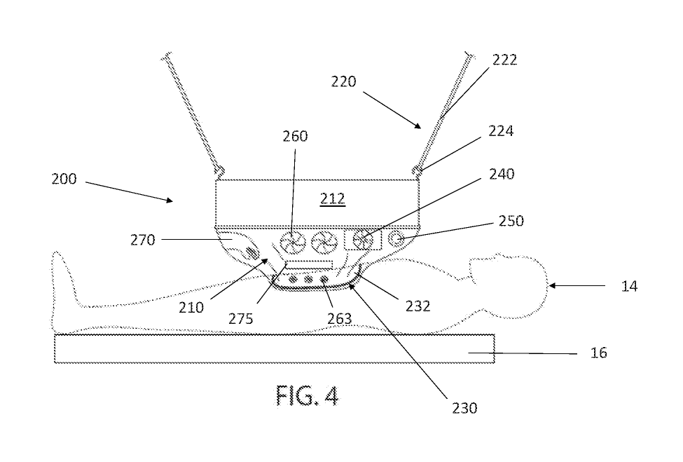

In another embodiment, the sterile enclosure may be structured for a single site attachment to any portion of the subject's body. For example, as shown in FIG. 4, a sterile environment device 200 may include similar features as described for device 100 of FIG. 1, including an enclosure material 210, a subject attachment region 230, at least one gas or air intake port 240, at least one gas or air exhaust port 250, and at least one access port, such as ports 260 and/or integrated gloves 270. Further, device 200 may include line ports 263, portals 275 or any other component as previously described for device 100. Like device 100 of FIG. 1, it should be appreciated that device 200 may also further include other structural features or openings for suitably introducing additional equipment, gases and the like into the sterile environment, as would be desired, such as ports and connectors for connecting lines for irrigation fluid, vacuum aspiration, and delivering power for ultrasonic phacoemulsification and a vitrectomy handpiece. As contemplated herein, device 200 may further include one or more rigid wall panels 212 to provide additional structure and define the open space within the formed sterile environment enclosure. Further, device 200 may include a suspension mechanism 220 for suspending device 200 above the subject. In one embodiment, suspension mechanism 220 may include one or more attachment hooks 224 and tension lines 222 that may be anchored to either a portion of the room (such as the ceiling), or to an extended arm from the room wall or from a separate standing equipment component. It should be appreciated that there is no limitation in the suspension mechanism 220 of device 200, provided that device 200 may be suitably positioned above any desired portion of the subject's body to be treated.

The disclosures of each and every patent, patent application, and publication cited herein are hereby incorporated herein by reference in their entirety.

While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. A sterile enclosure for performing an ophthalmologic procedure, comprising:
a lightweight, flexible housing material forming an enclosure;
at least one equipment attachment region within the housing material;
at least one subject attachment region within the housing material;
at least one air intake port;
at least one air exhaust port; and
at least one user access port within the housing material.

2. The enclosure of claim 1, wherein the at least one air intake port further comprises a filter.

3. The enclosure of claim 1, wherein the at least one access port is a glove integrated with the housing material.

4. The enclosure of claim 1, wherein the at least one access port is a flexible septum having separable leaflets.

5. The enclosure of claim 1, wherein the flexible housing material includes a frame.

6. The enclosure of claim 1, wherein the equipment attachment region includes an adhesive perimeter for at least temporary attachment to the desired equipment component.

7. The enclosure of claim 1, wherein the subject attachment region includes an adhesive perimeter for at least temporary attachment around the treatment site of the subject.

8. The enclosure of claim 1, wherein the housing material enclosure is inflated with air.

9. The enclosure of claim 1, wherein a bactericidal gas is pumped into the housing material enclosure.

10. A sterile enclosure for performing a medical procedure, comprising:
a lightweight, flexible housing material forming an enclosure;
a suspension mechanism for suspending the enclosure above a subject;
at least one subject attachment region within the housing material;
at least one air intake port;
at least one air exhaust port; and
at least one user access port within the housing material.

11. The enclosure of claim 10, wherein the at least one air intake port further comprises a filter.

12. The enclosure of claim 10, wherein the at least one access port is a glove integrated with the housing material.

13. The enclosure of claim 10, wherein the at least one access port is a flexible septum having separable leaflets.

14. The enclosure of claim 10, wherein the flexible housing material includes a frame.

15. The enclosure of claim 10, wherein the subject attachment region includes an adhesive perimeter for at least temporary attachment around the treatment site of the subject.

16. The enclosure of claim 10, wherein the housing material enclosure is inflated with air.

17. The enclosure of claim 10, wherein a bactericidal gas is pumped into the housing material enclosure.

18. The enclosure of claim 10, wherein the suspension mechanism comprises a hook and tension line attached to an anchor point.

19. The enclosure of claim 10, wherein the housing material includes at least one rigid panel.

20. A system for creating a temporary sterile environment to perform an ophthalmologic procedure, comprising:
a lightweight, flexible housing material forming an enclosure;
at least one equipment attachment region within the housing material;
at least one subject attachment region within the housing material;
at least one user access port within the housing material; and
an air flow circuit for controlling the flow of air within the housing material enclosure.
